(19) United States
(12) Patent Application Publication
(10) Pub. No.: US 2025/0339100 A1
(43) Pub. Date: Nov. 6, 2025

(54) ELECTRICAL TRACES ALONG CORE WIRE FOR INTRALUMINAL PHYSIOLOGY SENSING GUIDEWIRE AND ASSOCIATED DEVICES, SYSTEMS, AND METHODS

(71) Applicant: KONINKLIJKE PHILIPS N.V., EINDHOVEN (NL)

(72) Inventor: Miranda TAYLOR, OCEANSIDE, CA (US)

(21) Appl. No.: 18/854,032

(22) PCT Filed: Apr. 4, 2023
(86) PCT No.: PCT/EP2023/058877

§ 371 Date: Oct. 3, 2024

Related U.S. Application Data
(60) Provisional application No. 63/328,838, filed on Apr. 8, 2022.

(51) Int. Cl. A61B 5/00; A61B 5/021; A61B 5/0215; A61B 5/026; A61M 25/09

(52) U.S. Cl. CPC A61B 5/6851; A61B 5/02141; A61B 5/0215; A61B 5/026; A61B 5/6876; A61M 25/09; A61B 2560/0462; A61B 2562/0247; A61B 2562/227; A61M 2025/09083; A61M 2025/09133; A61M 2025/09175; A61M 2205/0233; A61M 2205/3303; A61M 2205/3331; A61M 2210/12; A61M 2230/30

(57) ABSTRACT

An apparatus includes an intravascular guidewire that includes a flexible elongate member configured to be positioned within a blood vessel. The flexible elongate member includes a proximal portion and a distal portion. The distal portion includes a core wire. The intravascular guidewire includes a sensor disposed at the distal portion of the flexible elongate member. The intravascular guidewire includes a first conductive member disposed at the distal portion of the flexible elongate member and coupled to the sensor. The first conductive member includes a first electrical trace disposed along the core wire. The intravascular guidewire includes a second conductive member disposed at the proximal portion of the flexible elongate member. The second conductive member is coupled to the first conductive member such that the second conductive member is in electrical communication with the sensor.

### TECHNICAL FIELD

The subject matter described herein relates to intraluminal physiology sensing devices (e.g., an intravascular pressure sensing and/or flow sensing guidewire). For example, the intraluminal device may include electrical traces embedded in polymer along a length of a core wire for providing power and/or data communication between a distal sensor and a proximal electrical connector.

### BACKGROUND

Existing intravascular guidewires with a sensor have fine-gauge electrical wires that provide transmission of electrical signals for the sensor. Manufacture of such devices may include many steps that involve human operator or machine contact with the fine-gauge electrical wires. Because of their delicate nature, the fine-gauge electrical wires are prone to damage as a result of such contact. For example, the conductors themselves may break and the insulation may be damaged. This leads to poor or no electrical connectivity for the sensor. Additionally, the fine-gauge electrical wires typically have circular cross-section, which occupies space with the very small outer diameter of the guidewire.

The information included in this Background section of the specification, including any references cited herein and any description or discussion thereof, is included for technical reference purposes only and is not to be regarded as subject matter by which the scope of the disclosure is to be bound.

### SUMMARY

Disclosed are intraluminal physiology sensing devices (e.g., an intravascular pressure-sensing and/or flow-sensing guidewire) that include electrical traces for power and signal communication. The electrical traces may be printed conductive ink traces, which permit improved electrical/mechanical performance and reduce or eliminate damage associated with handling the fine-gauge electrical wires. Because conductive ink traces have a flatter profile, using printed conductive ink traces may allow for a larger outer diameter of the intravascular guidewire's distal core wire and thicker polymer coating. This may provide for lower electrical resistance/impedance along the length of the electro-mechanical device, as well as improving the straightness and torque response of intraluminal devices, while reducing manufacturing defects and reducing the complexity of the intraluminal device manufacturing process.

The electrical trace assembly disclosed herein has particular, but not exclusive, utility for intraluminal medical catheters, guidewires, or guide catheters.

In an exemplary aspect, an apparatus is provided. The apparatus includes an intravascular guidewire comprising: a flexible elongate member configured to be positioned within a blood vessel of a patient, wherein the flexible elongate member comprises a proximal portion, a distal portion, wherein the distal portion of the flexible elongate member comprises a core wire; a sensor disposed at the distal portion of the flexible elongate member, wherein the sensor is configured to obtain medical data related to the blood vessel while the flexible elongate member is positioned within the blood vessel; a first conductive member disposed at the distal portion of the flexible elongate member, wherein the first conductive member is coupled to the sensor, wherein the first conductive member comprises a first electrical trace disposed along the core wire; and a second conductive member disposed at the proximal portion of the flexible elongate member, wherein the second conductive member is coupled to the first conductive member such that the second conductive member is in electrical communication with the sensor.

In some aspects, the flexible elongate member further comprises a first layer of insulating material disposed over the core wire such that the insulating material is disposed between the core wire and the first electrical trace. In some aspects, the first electrical trace comprises a conductive ink trace printed directly on the first layer of insulating material. In some aspects, the flexible elongate member further comprises a second layer of insulating material disposed over the first electrical trace. In some aspects, in a cross-section, a perimeter of the first electrical trace is completely surrounded by at least one of the first insulating material or the second insulating material. In some aspects, the flexible elongate member further comprises a polymer coating disposed over the second insulating material. In some aspects, the flexible elongate member further comprises a hydrophilic coating disposed over the polymer coating. In some aspects, the first electrical trace is disposed along the core wire in a spiral pattern. In some aspects, the first conductive member further comprises a second electrical trace disposed along the core wire in the spiral pattern, wherein the flexible elongate member further comprises an insulating material disposed over the first electrical trace and the second electrical trace, wherein adjacent windings of the first electrical trace and the second electrical trace are spaced from one another by only the insulating material. In some aspects, the first conductive member further comprises a second electrical trace disposed along the core wire in the spiral pattern, wherein the flexible elongate member further comprises: an insulating material disposed over the first electrical trace and the second electrical trace; and a polymer coating disposed over the insulating material, wherein adjacent windings of the first electrical trace and the second electrical trace are spaced from one another by the insulating material and the polymer coating. In some aspects, the first conductive member further comprises a second electrical trace disposed along the core wire in the spiral pattern, wherein the flexible elongate member further comprises a polymer coating disposed over the first electrical trace and the second electrical trace, wherein first portions of the insulating material are aligned with adjacent windings of the first electrical trace, wherein the adjacent windings of the first electrical trace are spaced from one another by a second portion of the insulating material, wherein the second portion of the insulating material directly contacts the core wire and the first portion of the insulating material does not directly contact the core wire. In some aspects, the first electrical trace comprises a conductive ink trace, wherein the intravascular guidewire further comprises a third conductive member and at least one conductive pad, wherein the first conductive member is coupled to the third conductive member via the at least one conductive pad, wherein the third conductive member is coupled to the at least one conductive pad, and wherein the conductive ink trace is printed onto the at least one conductive pad, wherein the third conductive member is directly coupled to the sensor. In some aspects, the first conductive member is disposed between the second conductive member and the third conductive member. In some aspects, the first electrical trace comprises a conductive ink trace, wherein the intravascular guidewire further comprises at least one conductive pad, wherein the second conductive member is coupled to the first conductive member via the at least one conductive pad, wherein the second conductive member is coupled to the conductive pad, and wherein the conductive ink trace is printed onto the at least one conductive pad. In some aspects, the sensor comprises at least one of a pressure sensor or a flow sensor.

In an exemplary aspect, an apparatus is provided. The apparatus includes an intravascular guidewire comprising: a flexible elongate member configured to be positioned within a blood vessel of a patient, wherein the flexible elongate member comprises a proximal portion, a distal portion, wherein the distal portion of the flexible elongate member comprises a distal core wire, and wherein the proximal portion of the flexible elongate member comprises a proximal core wire; at least one of a pressure sensor or a flow sensor disposed at the distal portion of the flexible elongate member and configured to obtain at least one of pressure data or flow data related to the blood vessel while the flexible elongate member is positioned within the blood vessel; a first conductive member disposed at the distal portion of the flexible elongate member, wherein the first conductive member is directly coupled to at least one of the pressure sensor or the flow sensor; a second conductive member disposed at the distal portion of the flexible elongate member, wherein the second conductive member comprises a plurality of conductive ink traces disposed along the core wire; and a third conductive member disposed at the proximal portion of the flexible elongate member, wherein the second conductive member is coupled to the first conductive member and the third conductive member such that the third conductive member is in electrical communication with at least one of the pressure sensor or the flow sensor.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the metal ink conductor assembly, as defined in the claims, is provided in the following written description of various embodiments of the disclosure and illustrated in the accompanying drawings.

### BRIEF DESCRIPTION OF THE DRAWINGS

Illustrative embodiments of the present disclosure will be described with reference to the accompanying drawings, of which:

FIG. 1 is a diagrammatic top view of an intravascular device, according to aspects of the present disclosure.

FIG. 2 is a diagrammatic side view of an intravascular sensing system that includes an intravascular device, according to aspects of the present disclosure.

FIG. 3A is a diagrammatic side view of a portion of an intravascular device with electrical traces disposed along a distal core wire, in accordance with at least one embodiment of the present disclosure.

FIG. 3B is a diagrammatic cross-sectional side view of electrical traces disposed along a distal core wire, in accordance with at least one embodiment of the present disclosure.

FIG. 4A is a diagrammatic side view of a portion of an intravascular device with electrical traces disposed along a distal core wire, in accordance with at least one embodiment of the present disclosure.

FIG. 4B is a diagrammatic cross-sectional side view of electrical traces disposed along a distal core wire, in accordance with at least one embodiment of the present disclosure.

### DETAILED DESCRIPTION

Disclosed is an electrical trace assembly that provides an improved electrical/mechanical performance for an intraluminal sensing device and that eliminates manufacturing issues associated with conductive filars. Multi-filar bundles (e.g., bifilar or trifilar) are groupings or multiple (e.g., two or three) conductors, with each conductor being surrounded by insulating coating. Manufacturing or assembly processes that require tensioning and over-coating of a multi-filar bundle may benefit from the electrical trace assembly of the present disclosure.

Replacing embedded filars with printed conductive ink traces enables the use of conductive materials that may be printed or applied directly onto one or components of a flexible elongate member, in place of separate trifilar leads being wrapped around these components. Printed conductive ink traces allow for a larger outside diameter (OD) distal core wire, thicker polymer coating, and eliminate damage/scrap due to trifilar handling. This may enable lower electrical resistance/impedance along the length of the electro-mechanical intraluminal sensing device. Because ribbons and/or multi-filar conductor bundles add stiffness and local torques to a guidewire or catheter, the straightness and torque response of the intraluminal device with conductive ink traces may be improved as well, which may lead to a reduction in mechanical “whipping” responses when the device is manipulated within intravascular anatomy. Whipping occurs when the distal portion of the guidewire does not smoothly rotate with the proximal portion of the guidewire (e.g., which is controlled by a user), instead the distal portion of the guidewire rapidly rotates inside the blood vessel to catch up to the rotational position of the proximal portion of the guidewire.

Other manufacturing issues may also be alleviated by using the electrical trace assembly of the present disclosure. For example, the process of holding multiple filars and/or flattened ribbon wires in tension while laying them down and overcoating with an insulating layer is very difficult. Some current processes are only capable of being performed with two flattened ribbon wires or multi-filar conductor bundles per device. Conversely, the present disclosure enables the implementation of as many conductive ink traces as desired. Furthermore, the processes used to create filars and flattened ribbon wires, and to embed them along the length of a device, are close to physical limits of the materials, due to the electrical/mechanical specifications of the electro-mechanical device. This creates design and sourcing limitations. The metal ink conductor assembly of the present disclosure reduces or eliminates these difficulties.

Example devices incorporating a multi-filar conductor bundle and/or conductive ribbons include intraluminal medical guidewire devices as described for example in U.S. Pat. No. 10,595,820 B2, U.S. Patent Publication Nos. 2014/0187874, 2016/0058977, and 2015/0273187, and in U.S. Provisional Patent Application No. 62/552,993 (filed Aug. 31, 2017), each of which is hereby incorporated by reference in its entirety as though fully set forth herein.

These descriptions are provided for exemplary purposes only and should not be considered to limit the scope of the metal ink conductor assembly. Certain features may be added, removed, or modified without departing from the spirit of the claimed subject matter.

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. Further, while the embodiments of the present disclosure may be described with respect to a blood vessel, it will be understood that the devices, systems, and methods described herein may be configured for use in any suitable anatomical structure or body lumen including a blood vessel, blood vessel lumen, an esophagus, eustachian tube, urethra, fallopian tube, intestine, colon, and/or any other suitable anatomical structure or body lumen. In other embodiments, the devices, systems, and methods described herein may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood vessels, chambers or other parts of the heart, and/or other systems of the body. In addition to natural structures, the device 102 may be used to examine man-made structures such as, but without limitation, heart valves, stents, shunts, filters, and other devices. For the sake of brevity, however, the numerous iterations of these combinations will not be described separately.

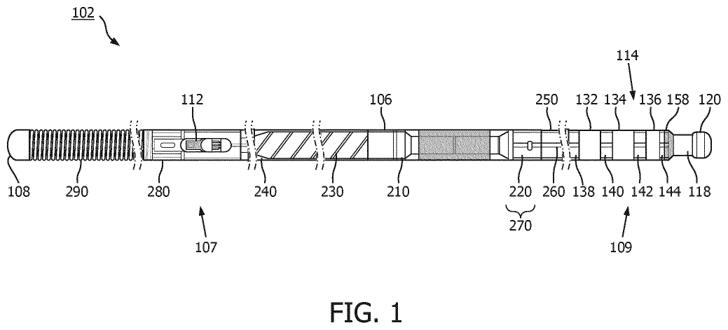

FIG. 1 is a diagrammatic top view of an intravascular device 102, according to aspects of the present disclosure. The intravascular device 102 may be an intravascular, intraluminal, or endoluminal guidewire, catheter, or guide catheter sized and shaped for positioning within a blood vessel of a patient. The intravascular device 102 may include a sensor 112. For example, the sensor 112 may be a pressure sensor configured to measure a pressure of blood flow within the vessel of the patient. The intravascular device 102 includes the flexible elongate member 106. The sensor 112 is disposed at the distal portion 107 of the flexible elongate member 106. The sensor 112 may be mounted at the distal portion 107 within a housing 280 in some embodiments. A flexible tip coil 290 extends between the housing 280 and the distal end 108. The connection portion 114 is disposed at the proximal portion 109 of the flexible elongate member 106. The connection portion includes the conductive portions 132, 134, 136. In some embodiments, the conductive portions 132, 134, 136 may be conductive ink that is printed and/or deposited around the flexible elongate member 106. In some embodiments, the conductive portions 132, 134, 136 may be conductive, metallic rings that are positioned around the flexible elongate member. The locking section 118 and knob or retention section 120 are disposed at the proximal portion 109 of the flexible elongate member 106.

The intravascular device 102 in FIG. 1 includes a distal core 210 and a proximal core 220. The distal core 210 and the proximal core 220 are metallic components forming part of the body of the intravascular device 102. For example, the distal core 210 and the proximal core 220 are flexible metallic rods that provide structure for the flexible elongate member 106. The diameter of the distal core 210 and the proximal core 220 may vary along its length.

In some embodiments, the intravascular device 102 comprises a distal assembly and a proximal assembly that are electrically and mechanically joined together, which results in electrical communication between the sensor 112 and the conductive portions 132, 134, 136. For example, pressure data obtained by the sensor 112 (in this example, sensor 112 is a pressure sensor) may be transmitted to the conductive portions 132, 134, 136. Control signals from a computer in communication with the intravascular device 102 may be transmitted to the sensor 112 via the conductive portions 132, 134, 136. The distal subassembly may include the distal core 210. The distal subassembly may also include the sensor 112, conductive members 230, and/or one or more layers of polymer/plastic 240 surrounding the conductive members 230 and the core 210. For example, the polymer/plastic layer(s) may protect the conductive members 230. The proximal subassembly may include the proximal core 220. The proximal subassembly may also include one or more layers of polymer layer(s) 250 (hereinafter polymer layer 250) surrounding the proximal core 220 and/or conductive ribbons 260 embedded within the one or more layers of polymer layer(s) 250. In some embodiments, the proximal subassembly and the distal subassembly may be separately manufactured. During the assembly process for the intravascular device 102, the proximal subassembly and the distal subassembly may be electrically and mechanically joined together. As used herein, flexible elongate member may refer to one or more components along the entire length of the intravascular device 102, one or more components of the proximal subassembly (e.g., including the proximal core 220, etc.), and/or one or more components the distal subassembly 210 (e.g., including the distal core 210, etc.).

In various embodiments, the intravascular device 102 may include one, two, three, or more core wires extending along its length. For example, a single core wire may extend substantially along the entire length of the flexible elongate member 106. In such embodiments, the locking section 118 and the knob or retention section 120 may be integrally formed at the proximal portion of the single core wire. The sensor 112 may be secured at the distal portion of the single core wire. In other embodiments, such as the embodiment illustrated in FIG. 1, the locking section 118 and the knob or retention section 120 may be integrally formed at the proximal portion of the proximal core 220. The sensor 112 may be secured at the distal portion of the distal core 210. The intravascular device 102 includes one or more conductive members 230 in communication with the electronic component 112. For example, the conductive members 230 may be one or more electrical wires that are directly in communication with the sensor 112. In some instances, the conductive members 230 are electrically and mechanically coupled to the sensor 112 by, e.g., soldering. In some instances, the conductive members 230 comprise two or three electrical wires (e.g., a bifilar cable or a trifilar cable). An individual electrical wire may include a bare metallic conductor surrounded by one or more insulating layers. The conductive members 230 may extend along the length of the distal core 210. For example, at least a portion of the conductive members 230 may be spirally wrapped around the distal core 210.

The intravascular device 102 includes one or more conductive ribbons 260 at the proximal portion of the flexible elongate member 106. The conductive ribbons 260 are embedded within polymer layer(s) 250. The conductive ribbons 260 are directly in communication with the conductive portions 132, 134, and/or 136. In some instances, the conductive members 230 are electrically and mechanically coupled to the sensor 112 by, e.g., soldering. In some instances, the conductive portions 132, 134, and/or 136 comprise conductive ink (e.g., metallic nano-ink, such as silver or gold nano-ink) that is deposited or printed directed over the conductive ribbons 260.

As described herein, electrical communication between the conductive members 230 and the conductive ribbons 260 may be established at the connection region 270 of the flexible elongate member 106. By establishing electrical communication between the conductive members 230 and the conductive ribbons 260, the conductive portions 132, 134, 136 may be in electrically communication with the sensor 112.

In some embodiments represented by FIG. 1, intravascular device 102 includes the locking section 118 and the knob or retention section 120. To form the locking section 118, a machining process is necessary to remove the polymer layer 250 and the conductive ribbons 260 in the locking section 118 and to shape proximal core 220 in the locking section 118 to the desired shape. As shown in FIG. 1, the locking section 118 includes a reduced diameter while the knob or retention section 120 has a diameter substantially similar to that of proximal core 220 in the connection portion 114. In some instances, because the machining process removes conductive ribbons in locking section 118, proximal ends of the conductive ribbons 260 would be exposed to moisture and/or liquids, such as blood, saline solutions, disinfectants, and/or enzyme cleaner solutions, an insulation layer 158 is formed over the proximal end portion of the connection portion 114 to insulate the exposed conductive ribbons.

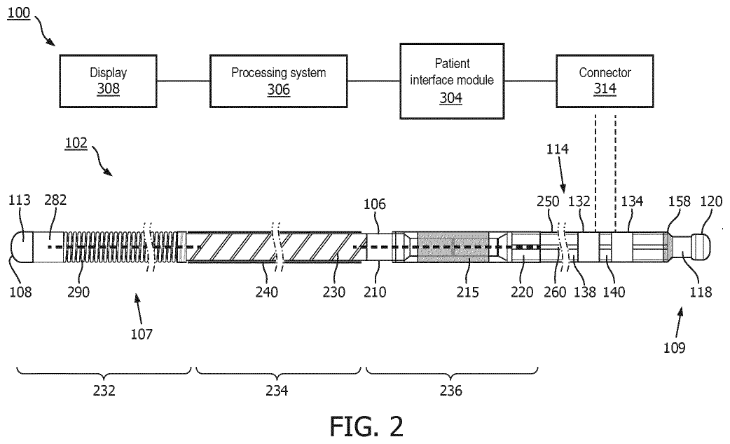

FIG. 2 is a diagrammatic side view of an intraluminal (e.g., intravascular) sensing system 100 that includes an intravascular device 102 comprising conductive members 230 (e.g., a multi-filar electrical conductor bundle) and conductive ribbons 260, according to aspects of the present disclosure. The intravascular device 102 may be an intravascular guidewire sized and shaped for positioning within a blood vessel of a patient. The intravascular device 102 includes a distal tip 108 and a sensor 113. For example, the sensor 113 may be a pressure sensor and/or flow sensor configured to measure a pressure of blood flow within the vessel of the patient, or another type of sensor including but not limited to a temperature or imaging sensor, or combination sensor measuring more than one property. For example, the flow data obtained by a flow sensor may be used to calculate physiological variables such as coronary flow reserve (CFR). The intravascular device 102 includes a flexible elongate member 106. The sensor 113 is disposed at a distal portion 107 of the flexible elongate member 106. The sensor 113 may be mounted at the distal portion 107 within a housing 282 in some embodiments. A flexible tip coil 290 extends distally from the housing 282 at the distal portion 107 of the flexible elongate member 106. A connection portion 114 located at a proximal end of the flexible elongate member 106 includes conductive portions 132, 134. In some embodiments, the conductive portions 132, 134 may be conductive ink that is printed and/or deposited around the connection portion 114 of the flexible elongate member 106. In some embodiments, the conductive portions 132, 134 are conductive, may be metallic bands or rings that are positioned around the flexible elongate member. A locking area is formed by a collar or locking section 118 and knob or retention section 120 are disposed at the proximal portion 109 of the flexible elongate member 106.

The intravascular device 102 in FIG. 2 includes core wire comprising a distal core 210 and a proximal core 220. The distal core 210 and the proximal core 220 are metallic components forming part of the body of the intravascular device 102. For example, the distal core 210 and the proximal core 220 may be flexible metallic rods that provide structure for the flexible elongate member 106. The distal core 210 and/or the proximal core 220 may be made of a metal or metal alloy. For example, the distal core 210 and/or the proximal core 220 may be made of stainless steel, Nitinol, nickel-cobalt-chromium-molybdenum alloy (e.g., MP35N), and/or other suitable materials. In some embodiments, the distal core 210 and the proximal core 220 are made of the same material. In other embodiments, the distal core 210 and the proximal core 220 are made of different materials. The diameter of the distal core 210 and the proximal core 220 may vary along their respective lengths. A joint between the distal core 210 and proximal core 220 is surrounded and contained by a hypotube 215. The sensor 113 may in some cases be positioned at a distal end of the distal core 210.

In some embodiments, the intravascular device 102 comprises a distal subassembly and a proximal subassembly that are electrically and mechanically joined together, which creates an electrical communication between the sensor 113 and the conductive portions 132, 134. For example, flow data obtained by the sensor 113 (in this example, sensor 113 is a flow sensor) may be transmitted to the conductive portions 132, 134. In an exemplary embodiment, the sensor 113 is a single ultrasound transducer element. The transducer element emits ultrasound signals and receives echoes. The transducer element generates electrical signals representative of the echoes. The signal carrying filars carry this electrical signal from the sensor at the distal portion to the connector at the proximal portion. The processing system 306 processes the electrical signals to extract the flow velocity of the fluid.

Control signals from a processing system 306 (e.g., a processor circuit of the processing system 306) in communication with the intravascular device 102 may be transmitted to the sensor 113 via a connector 314 that is attached to the conductive portions 132, 134. The distal subassembly may include the distal core 210. The distal subassembly may also include the sensor 113, the conductive members 230, and/or one or more layers of insulative polymer/plastic 240 surrounding the conductive members 230 and the core 210. For example, the polymer/plastic layer(s) may insulate and protect the conductive members of the multi-filar cable or conductor bundle 230. The proximal subassembly may include the proximal core 220. The proximal subassembly may also include one or more polymer layers 250 (hereinafter polymer layer 250) surrounding the proximal core 220 and/or conductive ribbons 260 embedded within the one or more insulative and/or protective polymer layer 250. In some embodiments, the proximal subassembly and the distal subassembly are separately manufactured. During the assembly process for the intravascular device 102, the proximal subassembly and the distal subassembly may be electrically and mechanically joined together. As used herein, flexible elongate member 106 may refer to one or more components along the entire length of the intravascular device 102, one or more components of the proximal subassembly (e.g., including the proximal core 220, etc.), and/or one or more components the distal subassembly (e.g., including the distal core 210, etc.). Accordingly, flexible elongate member 106 may refer to the combined proximal and distal subassemblies described above. The joint between the proximal core 220 and distal core 210 is surrounded by the hypotube 215.

In various embodiments, the intravascular device 102 may include one, two, three, or more core wires extending along its length. For example, a single core wire may extend substantially along the entire length of the flexible elongate member 106. In such embodiments, a locking section 118 and a section 120 may be integrally formed at the proximal portion of the single core wire. The sensor 113 may be secured at the distal portion of the single core wire. In other embodiments, such as the embodiment illustrated in FIG. 2, the locking section 118 and the section 120 may be integrally formed at the proximal portion of the proximal core 220. The sensor 113 may be secured at the distal portion of the distal core 210. The intravascular device 102 includes one or more conductive members 230 (e.g., a multi-filar conductor bundle or cable) in communication with the sensor 113. For example, the conductive members 230 may be one or more electrical wires that are directly in communication with the sensor 113. In some instances, the conductive members 230 are electrically and mechanically coupled to the sensor 113 by, e.g., soldering. In some instances, the conductor bundle 230 comprises two or three electrical wires (e.g., a bifilar cable or a trifilar cable). An individual electrical wire may include a bare metallic conductor surrounded by one or more insulating layers. The conductive members 230 may extend along the length of the distal core 210. For example, at least a portion of the conductive members 230 may be spirally wrapped around the distal core 210, minimizing or eliminating whipping of the distal core within tortuous anatomy.

The intravascular device 102 includes one or more conductive ribbons 260 at the proximal portion of the flexible elongate member 106. The conductive ribbons 260 are embedded within polymer layer 250. The conductive ribbons 260 are directly in communication with the conductive portions 132 and/or 134. In some instances, a multi-filar conductor bundle 230 is electrically and mechanically coupled to the sensor 113 by, e.g., soldering. In some instances, the conductive portions 132 and/or 134 comprise conductive ink (e.g., metallic nano-ink, such as copper, silver, gold, or aluminum nano-ink) that is deposited or printed directed over the conductive ribbons 260.

As described herein, electrical communication between the conductive members 230 and the conductive ribbons 260 may be established at the connection portion 114 of the flexible elongate member 106. By establishing electrical communication between the conductor bundle 230 and the conductive ribbons 260, the conductive portions 132, 134 may be in electrical communication with the sensor 113.

In some embodiments represented by FIG. 1, the intravascular device 102 includes a locking section 118 and knob or retention section 120. To form locking section 118, a machining process is used to remove polymer layer 250 and conductive ribbons 260 in locking section 118 and to shape proximal core 220 in locking section 118 to the desired shape. As shown in FIG. 1, locking section 118 includes a reduced diameter while knob or retention has a diameter substantially similar to that of proximal core 220 in the connection portion 114. In some instances, because the machining process removes conductive ribbons in locking section 118, proximal ends of the conductive ribbons 260 would be exposed to moisture and/or liquids, such as blood, saline solutions, disinfectants, and/or enzyme cleaner solutions, an insulation layer 158 is formed over the proximal end portion of the connection portion 114 to insulate the exposed conductive ribbons 260.

In some embodiments, a connector 314 provides electrical connectivity between the conductive portions 132, 134 and a patient interface monitor 304. The Patient Interface Monitor (PIM) 304 may in some cases connect to a console or processing system 306, which includes or is in communication with a display 308.

The system 100 may be deployed in a catheterization laboratory having a control room. The processing system 306 may be located in the control room. Optionally, the processing system 306 may be located elsewhere, such as in the catheterization laboratory itself. The catheterization laboratory may include a sterile field while its associated control room may or may not be sterile depending on the procedure to be performed and/or on the health care facility. In some embodiments, device 102 may be controlled from a remote location such as the control room, such that an operator is not required to be in close proximity to the patient.

The intraluminal device 102, PIM 304, and display 308 may be communicatively coupled directly or indirectly to the processing system 306. These elements may be communicatively coupled to the medical processing system 306 via a wired connection such as a standard copper multi-filar conductor bundle 230. The processing system 306 may be communicatively coupled to one or more data networks, e.g., a TCP/IP-based local area network (LAN). In other embodiments, different protocols may be utilized such as Synchronous Optical Networking (SONET). In some cases, the processing system 306 may be communicatively coupled to a wide area network (WAN).

The PIM 304 transfers the received signals to the processing system 306 where the information is processed and displayed (e.g., as physiology data in graphical, symbolic, or alphanumeric form) on the display 308. The console or processing system 306 may include a processor and a memory. The processing system 306 may be operable to facilitate the features of the intravascular sensing system 100 described herein. For example, the processor may execute computer readable instructions stored on the non-transitory tangible computer readable medium.

The PIM 304 facilitates communication of signals between the processing system 306 and the intraluminal device 102. The PIM 304 may be communicatively positioned between the processing system 306 and the intraluminal device 102. In some embodiments, the PIM 304 performs preliminary processing of data prior to relaying the data to the processing system 306. In examples of such embodiments, the PIM 304 performs amplification, filtering, and/or aggregating of the data. In an embodiment, the PIM 304 also supplies high- and low-voltage DC power to support operation of the intraluminal device 102 via the conductive members 230.

A multi-filar cable or transmission line bundle 230 may include a plurality of conductors, including one, two, three, four, five, six, seven, or more conductors. In the example shown in FIG. 2, the multi-filar conductor bundle 230 includes two straight portions 232 and 236, where the multi-filar conductor bundle 230 lies parallel to a longitudinal axis of the flexible elongate member 106, and a spiral portion 234, where the multi-filar conductor bundle 230 is wrapped around the exterior of the flexible elongate member 106 and then overcoated with an insulative and/or protective polymer 240. Communication, if any, along the multi-filar conductor bundle 230 may be through numerous methods or protocols, including serial, parallel, and otherwise, where one or more filars of the bundle 230 carry signals. One or more filars of the multi-filar conductor bundle 230 may also carry direct current (DC) power, alternating current (AC) power, or serve as a ground connection.

The display or monitor 308 may be a display device such as a computer monitor or other type of screen. The display or monitor 308 may be used to display selectable prompts, instructions, and visualizations of imaging data to a user. In some embodiments, the display 308 may be used to provide a procedure-specific workflow to a user to complete an intraluminal imaging procedure.

Before continuing, it should be noted that the examples described above are provided for purposes of illustration and are not intended to be limiting. Other devices and/or device configurations may be utilized to carry out the operations described herein.

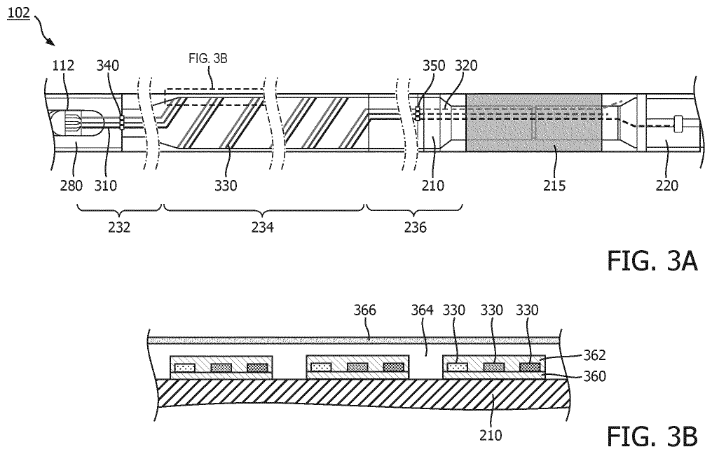

FIG. 3A is a diagrammatic side view of a portion of the intravascular device 102 comprising electrical traces 330 disposed along the distal core wire 210, in accordance with at least one embodiment of the present disclosure. Visible are the proximal core wire 220, the distal core wire 210, joined by a hypotube 215. At the distal end of the distal core wire 210 is a sensor 112 that may be fully or partially enclosed within housing 280. Also visible are conductive pathways 310 and 320 and electrical traces 330, where electrical traces 330 are disposed and/or extended in a longitudinal direction between conductive pathways 310 and 320. In some embodiments, the electrical traces 330 are distinct electrical traces in that they are physically and electrically separate from one another, but structurally made of a same material. In other embodiments, the electrical traces 330 may structurally be made of different materials. In some embodiments, one sensor 113 may be at the distal end of the intravascular device 102, and a different sensor 112 may be spaced from the distal end.

The conductive pathways 310 and 320 and electrical traces 330 electrically connect the sensor 112 with the conductive regions or bands 132, 134, and 136 on the proximal core wire 220 (FIGS. 1 and 2). Electrical traces 330 may be conductive ink traces that are disposed onto the distal core wire 210 by, e.g. printing. A distal end of conductive pathways 310 is coupled and/or joined to the sensor 112. A proximal end of conductive pathways 310 is coupled and/or joined to conductive pads 340. In some instances, the coupling between the distal end of the conductive pathways 310 and the sensor 112 and the coupling between the proximal end of the conductive pathways 310 and the conductive pads 340 may be an electrical coupling, a mechanical coupling, and/or an electrical and mechanical coupling formed by, e.g., soldering, ultrasonic welding, conductive adhesive, electrical bonding, etc. A distal end of electrical traces 330 is electrically and mechanically coupled to the conductive pads 340 by, e.g., printing onto the conductive pads 340. In some instances, conductive pads 340 may be omitted such that the conductive pathways 310 and the electrical traces 330 are the same electrical trace that extends from the sensor 112 and over the distal core 210. For example, conductive pathways 310 and 330 may refer to different portions, segments, or lengths of the same component. In such instances, the electrical traces 330 are printed directly onto the conductive pads of the sensor 112. Examples of directly coupling an electrical trace to a sensor are described in App. No. 63/328,330, filed Apr. 7, 2022, and titled “Continuous Electrical Trace In Intraluminal Device And Associated Devices, Systems, And Methods”. The conductive pathways 310 and a distal portion of the electrical traces 330 are included in a straight region 232, which is a region where all or part of the electrical traces 330 and/or the conductive pathways 310 extend linearly in a longitudinal direction. The electrical traces 330 also includes a spiral region 234 where electrical traces 330 wrap around the distal core wire 210 in a spiral pattern. A proximal end of electrical traces 330 is electrically and mechanically coupled to the conductive pads 350 by, e.g., printing onto the conductive pads 350. A distal end of conductive pathways 320 is coupled and/or joined to conductive pads. In some instances, the coupling between the distal end of the conductive pathways 320 and the conductive pads 350 may be an electrical coupling, a mechanical coupling, and/or an electrical and mechanical coupling formed by, e.g., soldering, ultrasonic welding, conductive adhesive, electrical bonding, etc. The conductive pathways 320 and a proximal portion of the electrical traces 330 are included in a straight region 236, which is a region where all or part of the electrical traces 330 and/or the conductive pathways 320 extend linearly in a longitudinal direction. A proximal end of the conductive pathways 320 then extends past the hypotube 215 (e.g., within an interior of the hypotube 215 or outside of the hypotube 215) before coupling to the conductive regions 132, 134, and 136 on the proximal core wire 220 (FIGS. 1 and 2).

Aspects of the present disclosure may include features described in App. No. 63/330,427, filed Apr. 13, 2022, and titled “Flex Circuit For Electrical Connection In Intraluminal Device And Associated Devices, Systems, And Methods”, App. No. 63/332,763, filed Apr. 20, 2022, and titled “Flex Circuit Around Core Wire In Intraluminal Device And Associated Devices, Systems, And Methods”, and App. No. 63/228,330, filed Apr. 7, 2022, and titled “Continuous Electrical Trace In Intraluminal Device And Associated Devices, Systems, And Methods”, the entirety of each of which is hereby incorporated by reference herein.

The intravascular device 102 may include any suitable quantity of conductors in the conductive pathways 310 and 320, e.g., two, three, four, five, or more. The intravascular device 102 may include any suitable quantity of electrical traces 330, include two, three, four, five, or more. In some embodiments, the intravascular device 102 includes the same quantity of conductors in the conductive pathways 310 and 320 as electrical traces 330. In some embodiments, a communication line is established between one of conductive pathways 310, one of electrical traces 330, one of conductive pathways 320, and one of conductive ribbons 260 that are electrically coupled/in communication with one another. In some embodiments, there are two or more communication lines for, e.g., power (positive, negative, ground), signal/data transmission.

FIG. 3B is a diagrammatic cross-sectional side view of the electrical traces 330 disposed and/or extended along the distal core wire 210, in accordance with at least one embodiment of the present disclosure. In particular, FIG. 3B is a cross-sectional view of a portion of the intravascular device 102 that is identified in FIG. 3A. A first flexible, insulative material 360 is disposed directly over and in direct contact with the distal core wire 210. In some embodiments, the insulative material 360 is provided only in areas where electrical traces 330 will be disposed. Electrical traces 330 are then disposed directly onto the first insulative material 360. Electrical traces 330 may be conductive ink traces that are disposed onto the distal core wire 210 by, e.g. printing, depositing, etc. A second flexible, insulative material 362 is disposed directly over the electrical traces 330. In some embodiments, the insulative material is provided only in areas where electrical traces 330 are disposed. As is shown in FIG. 3B, a perimeter of each electrical traces 330 is completely surrounded by the first flexible, insulative material 360 and/or the second flexible, insulative material 362. In some embodiments, the first flexible, insulative material 360 and the second flexible, insulative material 362 may be structurally made of a same material. In other embodiments, the first flexible, insulative material 360 and the second flexible, insulative material 362 may structurally be made of different materials. Similarly, the polymer coating 364 may be a same material or a different material than the first flexible, insulative material 360 and the second flexible, insulative material 362. The first flexible, insulative material 360 or the second flexible, insulative material 362 is aligned in a longitudinal direction with adjacent windings of the electrical traces 330. The second flexible, insulative material 362 are then overcoated with a polymer coating 364. Electrical traces 330 are spaced from one another longitudinally by only the second flexible, insulative material 362. In FIG. 3B, the conductive traces 330 are grouped together by the first flexible, insulative material 360 and the second flexible, insulative material 362. Each winding includes all three of the conductive traces 330. Each grouping is spaced by the polymer coating 364. Polymer coating 364 is overcoated with hydrophilic coating 366. The first insulative material 360, the electrical traces 330, the second insulative material 360, polymer coating 364, and the hydrophilic coating 366 have respective thicknesses in the radial direction and respective lengths in the longitudinal direction.

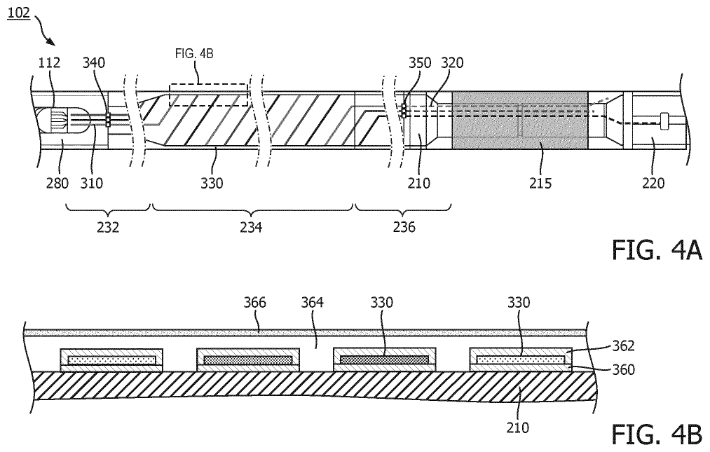

FIG. 4A is a diagrammatic side view of a portion of the intravascular device 102 comprising the electrical traces 330 disposed along the distal core wire 210, in accordance with at least one embodiment of the present disclosure. FIG. 4B is a diagrammatic cross-sectional side view of the electrical traces 330 disposed along the distal core wire 210, in accordance with at least one embodiment of the present disclosure. In particular, FIG. 4B is a cross-sectional side view of a portion of the intravascular device 102 that is identified in FIG. 4A. FIGS. 4A and 4B include features similar to those described in FIGS. 3A and 3B. In the embodiment of FIGS. 4A and 4B, the conductive traces 330 are not grouped together by the first flexible, insulative material 360 and the second flexible, insulative material 362. The windings of each electrical trace 330 is separate from one another compared to the grouping shown in FIG. 3B. A winding of the electrical trace 330 is spaced longitudinally from the next winding of the adjacent trace by both the second flexible, insulative material 362 and the polymer coating 364.

Accordingly, it may be seen that the electrical trace assembly advantageously reduces or eliminates the need for tensioning of multi-filar conductor bundles and/or conductive ribbons that may be used in the manufacture of small electronic devices such as intravascular medical catheters, guidewires, and guide catheters. This may tend to eliminate the risk of elongation and/or necking that may compromise the mechanical and electrical properties of a conductor, as well as reducing the risk of gaps or thin spots in the outer insulative coating. A number of variations are possible on the examples and embodiments described above. The electrical trace assembly could be applied to any product that involves electrical conductors to be embedded into composite subassemblies. However, unlike conductive ribbons, printed conductive ink traces may conform to the curvature of the core wire.

The logical operations making up the embodiments of the technology described herein are referred to variously as operations, steps, objects, elements, components, or modules. Furthermore, it should be understood that these may be arranged or performed in any order, unless explicitly claimed otherwise or a specific order is inherently necessitated by the claim language. It should further be understood that the described technology may be employed in single-use and multi-use electrical and electronic devices for medical or nonmedical use.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the metal ink conductor assembly. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary embodiments of the metal ink conductor assembly as defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter.

Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims.

What is claimed is:

1. An apparatus, comprising:
an intravascular guidewire comprising:
a flexible elongate member configured to be positioned within a blood vessel of a patient, wherein the flexible elongate member comprises a proximal portion, a distal portion, wherein the distal portion of the flexible elongate member comprises a core wire;
a sensor disposed at the distal portion of the flexible elongate member, wherein the sensor is configured to obtain medical data related to the blood vessel while the flexible elongate member is positioned within the blood vessel;
a first conductive member disposed at the distal portion of the flexible elongate member, wherein the first conductive member is coupled to the sensor, wherein the first conductive member comprises a first electrical trace disposed along the core wire; and
a second conductive member disposed at the proximal portion of the flexible elongate member,
wherein the second conductive member is coupled to the first conductive member such that the second conductive member is in electrical communication with the sensor.

2. The apparatus of claim 1, wherein the flexible elongate member further comprises a first layer of insulating material disposed over the core wire such that the insulating material is disposed between the core wire and the first electrical trace.

3. The apparatus of claim 1, wherein the first electrical trace comprises a conductive ink trace printed directly on the first layer of insulating material.

4. The apparatus of claim 2, wherein the flexible elongate member further comprises a second layer of insulating material disposed over the first electrical trace.

5. The apparatus of claim 4, wherein, in a cross-section, a perimeter of the first electrical trace is completely surrounded by at least one of the first insulating material or the second insulating material.

6. The apparatus of claim 4, wherein the flexible elongate member further comprises a polymer coating disposed over the second insulating material.

7. The apparatus of claim 6, wherein the flexible elongate member further comprises a hydrophilic coating disposed over the polymer coating.

8. The apparatus of claim 1, wherein the first electrical trace is disposed along the core wire in a spiral pattern.

9. The apparatus of claim 8,
wherein the first conductive member further comprises a second electrical trace disposed along the core wire in the spiral pattern,
wherein the flexible elongate member further comprises an insulating material disposed over the first electrical trace and the second electrical trace,
wherein adjacent windings of the first electrical trace and the second electrical trace are spaced from one another by only the insulating material.

10. The apparatus of claim 8,
wherein the first conductive member further comprises a second electrical trace disposed along the core wire in the spiral pattern,
wherein the flexible elongate member further comprises:
an insulating material disposed over the first electrical trace and the second electrical trace; and
a polymer coating disposed over the insulating material,
wherein adjacent windings of the first electrical trace and the second electrical trace are spaced from one another by the insulating material and the polymer coating.

11. The apparatus of claim 8,
wherein the first conductive member further comprises a second electrical trace disposed along the core wire in the spiral pattern,
wherein the flexible elongate member further comprises a polymer coating disposed over the first electrical trace and the second electrical trace,
wherein first portions of the insulating material are aligned with adjacent windings of the first electrical trace,
wherein the adjacent windings of the first electrical trace are spaced from one another by a second portion of the insulating material,
wherein the second portion of the insulating material directly contacts the core wire and the first portion of the insulating material does not directly contact the core wire.

12. The apparatus of claim 1,
wherein the first electrical trace comprises a conductive ink trace,
wherein the intravascular guidewire further comprises a third conductive member and at least one conductive pad,
wherein the first conductive member is coupled to the third conductive member via the at least one conductive pad,
wherein the third conductive member is coupled to the at least one conductive pad, and wherein the conductive ink trace is printed onto the at least one conductive pad,
wherein the third conductive member is directly coupled to the sensor.

13. The apparatus of claim 12, wherein the first conductive member is disposed between the second conductive member and the third conductive member.

14. The apparatus of claim 1,
wherein the first electrical trace comprises a conductive ink trace,
wherein the intravascular guidewire further comprises at least one conductive pad,
wherein the second conductive member is coupled to the first conductive member via the at least one conductive pad,
wherein the second conductive member is coupled to the conductive pad, and wherein the conductive ink trace is printed onto the at least one conductive pad.

15. The apparatus of claim 1, wherein the sensor comprises at least one of a pressure sensor or a flow sensor.

16. An apparatus, comprising:
an intravascular guidewire comprising:
a flexible elongate member configured to be positioned within a blood vessel of a patient, wherein the flexible elongate member comprises a proximal portion, a distal portion, wherein the distal portion of the flexible elongate member comprises a distal core wire, and wherein the proximal portion of the flexible elongate member comprises a proximal core wire;
at least one of a pressure sensor or a flow sensor disposed at the distal portion of the flexible elongate member and configured to obtain at least one of pressure data or flow data related to the blood vessel while the flexible elongate member is positioned within the blood vessel;
a first conductive member disposed at the distal portion of the flexible elongate member, wherein the first conductive member is coupled to at least one of the pressure sensor or the flow sensor, wherein the first conductive member comprises a plurality of conductive ink traces disposed along the core wire; and
a second conductive member disposed at the proximal portion of the flexible elongate member,
wherein the second conductive member is coupled to the first conductive member such that the second conductive member is in electrical communication with at least one of the pressure sensor or the flow sensor.
